(19) United States
(12) Patent Application Publication
(10) Pub. No.: US 2024/0363222 A1
(43) Pub. Date: Oct. 31, 2024

(54) ARTIFICIAL INTELLIGENCE SYSTEMS AND METHODS FOR GENERATING EDUCATIONAL INQUIRY RESPONSES FROM BIOLOGICAL EXTRACTIONS

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/767,306

(22) Filed: Jul. 9, 2024

Related U.S. Application Data
(63) Continuation-in-part of application No. 16/825,098, filed on Mar. 20, 2020, now Pat. No. 12,045,696.

(51) Int. Cl. G16H 20/60; G06N 20/00

(52) U.S. Cl. CPC G16H 20/60; G06N 20/00

(57) ABSTRACT

An artificial intelligence system for generating educational inquiry responses from biological extractions and methods related thereto include a computing device designed and configured to retrieve a biological extraction pertaining to a user, identify, based on the biological extraction, at least a nutritional need of the user, receive at least an educational inquiry including a nutrition-related educational inquiry, select, based on the at least an educational inquiry, at least a machine-learning model, wherein selecting the at least a machine-learning model includes receiving biological extraction training data correlating exemplary biological extractions to exemplary nutritional support resources and training the at least a machine-learning model using the biological extraction training data, and generate, using the at least a machine-learning model and the at least a nutritional need of the user, an inquiry response including a plurality of nutritional support resources.

### CROSS-REFERENCE TO RELATED APPLICATIONS

This application is a continuation-in-part of U.S. Non-provisional application Ser. No. 16/825,098 filed on Mar. 20, 2020, now U.S. Pat. No. 12,045,696, issued on Jul. 23, 2024, and entitled “ARTIFICIAL INTELLIGENCE SYSTEMS AND METHODS FOR GENERATING EDUCATIONAL INQUIRY RESPONSES FROM BIOLOGICAL EXTRACTIONS”, the entirety of which is incorporated herein by reference.

### FIELD OF THE INVENTION

The present invention generally relates to the field of artificial intelligence. In particular, the present invention is directed to artificial intelligence systems and methods for generating educational inquiry responses from biological extractions.

### BACKGROUND

Efficient routing of inquiries to responses regarding educational requests remains elusive, owing to the divergent criteria according to which suitability of such routing may be adjudged. A resulting lack of specificity may end in dissatisfaction with resulting outputs.

### SUMMARY OF THE DISCLOSURE

In an aspect, an artificial intelligence system for generating educational inquiry responses from biological extractions is described. System includes a computing device designed and configured to retrieve a biological extraction pertaining to a user, wherein the biological extraction includes nutrition-related physiological data, identify, based on the biological extraction, at least a nutritional need of the user, wherein the at least a nutritional need of the user is determined using at least an assessment, receive at least an educational inquiry including a nutrition-related educational inquiry, select, based on the at least an educational inquiry, at least a machine-learning model, wherein selecting the at least a machine-learning model includes receiving biological extraction training data correlating exemplary biological extractions to exemplary nutritional support resources and training the at least a machine-learning model using the biological extraction training data, and generate, using the at least a machine-learning model and the at least a nutritional need of the user, an inquiry response including a plurality of nutritional support resources.

In another aspect, an artificial intelligence-based method for generating educational inquiry responses from biological extractions is described. Method includes retrieving biological extraction pertaining to user, wherein the biological extraction includes nutrition-related physiological data, identifying, based on the biological extraction, at least a nutritional need of the user, wherein the at least a nutritional need of the user is determined using at least an assessment, receiving at least an educational inquiry including nutrition-related educational inquiry, selecting, based on the at least an educational inquiry, at least a machine-learning model, wherein selecting the at least a machine-learning model includes receiving biological extraction training data correlating exemplary biological extractions to exemplary nutritional support resources and training the at least a machine-learning model using the biological extraction training data, and generating, using the at least a machine-learning model and the at least a nutritional need of the user, an inquiry response including a plurality of nutritional support resources.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of an artificial intelligence system for generating educational inquiry responses from biological extractions;

FIG. 2 is a block diagram illustrating an exemplary embodiment of a user database;

FIG. 3 is a flow diagram illustrating an exemplary embodiment of an artificial intelligence method of generating educational inquiry responses from biological extractions; and

FIG. 4 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Embodiments disclosed herein use machine-learning processes to select a response to an inquiry regarding education, based upon a biological extraction. Selection of machine-learning processes may be performed as a function of educational inquiries; selection may be performed via classification.

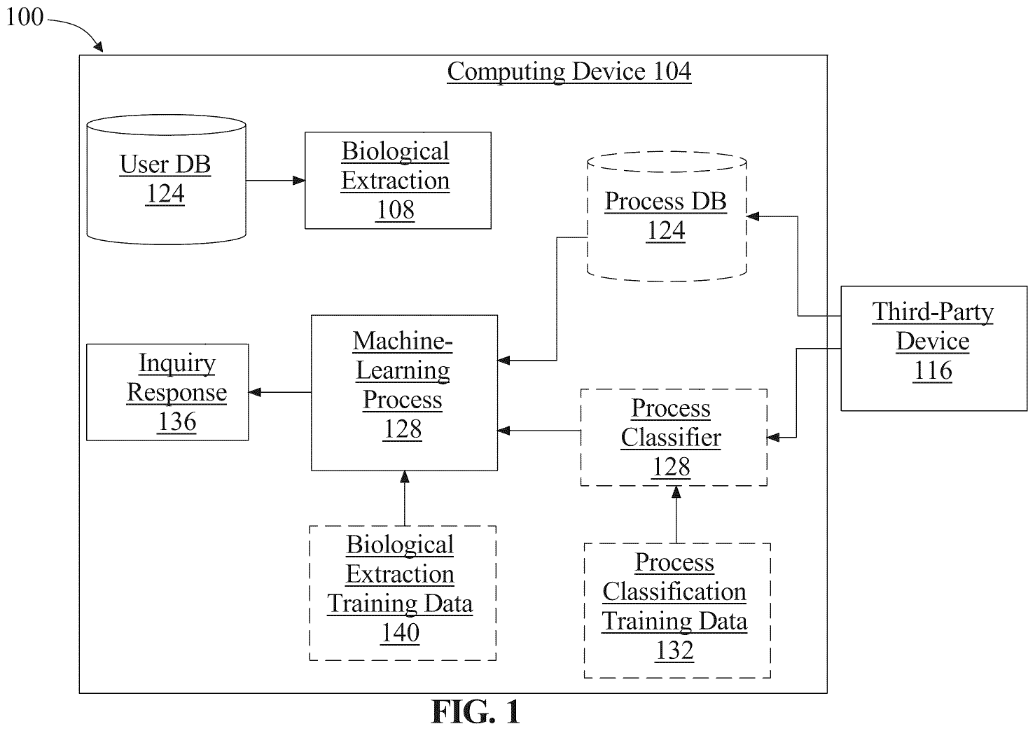

Referring now to FIG. 1, an exemplary embodiment of an artificial intelligence system 100 for generating educational inquiry responses from biological extractions 108 is illustrated. System includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices 104 in a first location and a second computing device 104 or cluster of computing devices 104 in a second location. Computing device 104 may include one or more computing devices 104 dedicated to data storage, security, distribution of traffic for load balancing, and the like. Computing device 104 may distribute one or more computing tasks as described below across a plurality of computing devices 104 of computing device 104, which may operate in parallel, in series, redundantly, or in any other manner used for distribution of tasks or memory between computing devices 104. Computing device 104 may be implemented using a “shared nothing” architecture in which data is cached at the worker, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 may be configured to receive a biological extraction 108 pertaining to a user. A “biological extraction”, as used in this disclosure is an element of data including at least an element of user physiological data. As used in this disclosure, “physiological data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, physiological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss. In some cases, physiological data may include nutrition-related physiological data pertaining to cases such as without limitation malnutrition including malnutrition and overnutrition, imbalanced nutrition and diet, as well as medical cases/risk factors stemming directly or indirectly therefrom, such as high blood pressure due to high salt intake, high blood sugar level due to overconsumption of added sugar, high cholesterol level due to overconsumption of fried food, certain vitamin deficiencies due to overconsumption of processed food, or the like.

With continued reference to FIG. 1, physiological data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological data may include, without limitation, immune function data such as Interleukine-6 (IL-6), TNF-alpha, systemic inflammatory cytokines, and the like.

Continuing to refer to FIG. 1, physiological data may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological data may include measures of glucose metabolism such as fasting glucose levels and/or hemoglobin A1-C(HbA1c) levels. Physiological data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological data may include measures of estimated glomerular filtration rate (eGFR). Physiological data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological data may include antinuclear antibody levels. Physiological data may include aluminum levels. Physiological data may include arsenic levels. Physiological data may include levels of fibrinogen, plasma cystatin C, and/or brain natriuretic peptide.

Continuing to refer to FIG. 1, physiological data may include measures of lung function such as forced expiratory volume, one second (FEV-1) which measures how much air can be exhaled in one second following a deep inhalation, forced vital capacity (FVC), which measures the volume of air that may be contained in the lungs. Physiological data may include a measurement of blood pressure, including without limitation systolic and diastolic blood pressure. Physiological data may include a measure of waist circumference. Physiological data may include body mass index (BMI). Physiological data may include one or more measures of bone mass and/or density such as dual-energy x-ray absorptiometry. Physiological data May include one or more measures of muscle mass. Physiological data may include one or more measures of physical capability such as without limitation measures of grip strength, evaluations of standing balance, evaluations of gait speed, pegboard tests, timed up and go tests, and/or chair rising tests.

Still viewing FIG. 1, physiological data may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological data may include one or more evaluations of sensory ability, including measures of audition, vision, olfaction, gustation, vestibular function and pain.

Continuing to refer to FIG. 1, physiological data may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices 104; for instance, user patterns of purchases, including electronic purchases, communication such as via chatrooms or the like, any textual, image, video, and/or data produced by the subject, any textual image, video and/or other data depicting and/or describing the subject, or the like. Any psychological data and/or data used to generate psychological data may be analyzed using machine-learning and/or language processing module as described in this disclosure. As a non-limiting example, biological extraction 108 may include a psychological profile; the psychological profile may be obtained utilizing a questionnaire performed by the user.

Still referring to FIG. 1, physiological data may include genomic data, including deoxyribonucleic acid (DNA) samples and/or sequences, such as without limitation DNA sequences or other genetic sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological data may include proteomic data, which as used herein is data describing all proteins produced and/or modified by an organism, colony of organisms, or system of organisms, and/or a subset thereof. Physiological data may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological data of a person, as described in further detail below.

With continuing reference to FIG. 1, physiological data may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals or the like, or any other user-entered data that a user may provide to a medical professional when seeking treatment and/or evaluation, and/or in response to medical intake papers, questionnaires, questions from medical professionals, or the like. Physiological data may include any physiological data, as described above, describing any multicellular organism living in or on a person including any parasitic and/or symbiotic organisms living in or on the persons; non-limiting examples may include mites, nematodes, flatworms, or the like. Examples of physiological data described in this disclosure are presented for illustrative purposes only and are not meant to be exhaustive.

With continued reference to FIG. 1, in some cases, biological extraction 108 may include at least a user behavior or one or more behavioral indicators or patterns pertaining to the user, consistent with details described below in this disclosure. As a nonlimiting example, user behavior may include one or more user preferences, such as preferred frequency of meals, preferred flavors or tastes. As another nonlimiting example, user behavior may include one or more user lifestyles, such as work schedules, exercise habits, among others. As another nonlimiting example, user behavior may include one or more temporal attributes, such as dietary routines. Dietary routines may include one or more preferred times of the day to consume a certain food and/or its associated frequency of consumption on a daily, weekly, or monthly basis, among others. As a nonlimiting example, dietary routine may include a routine of dining at home on Mondays, Wednesdays, and Fridays and dining out on Tuesdays and Thursdays. In some cases, biological extraction may include at least a digestive sensitivity pertaining to a user. Digestive sensitivity may include cases such as without limitation lactose intolerance, gluten intolerance, among others, as described below in this disclosure. In some cases, user behavior may include one or more user goals, such as weight loss, reduced hypertension, a reduced cholesterol level, a reduced stress level, an improvement in depressive disorders, or any similar metric that may signify an improved well-being of an individual.

With continued reference to FIG. 1, physiological data may include, without limitation any result of any medical test, physiological assessment, cognitive assessment, psychological assessment, or the like. System 100 may receive at least a physiological data from one or more other devices after performance; system 100 may alternatively or additionally perform one or more assessments and/or tests to obtain at least a physiological data, and/or one or more portions thereof, on system 100. For instance, at least physiological data may include or more entries by a user in a form or similar graphical user interface object; one or more entries may include, without limitation, user responses to questions on a psychological, behavioral, personality, or cognitive test. For instance, at least a server may present to user a set of assessment questions designed or intended to evaluate a current state of mind of the user, a current psychological state of the user, a personality trait of the user, or the like; at least a server may provide user-entered responses to such questions directly as at least a physiological data and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 1, in some cases, computing device 104 may be further configured to determine a current physical and mental state of user as a function of biological extraction 108 and the at least an assessment, compare the current physical and mental state of the user against an optimum physical and mental state, and generate a deviation metric as a function of the comparison. For the purposes of this disclosure, a “deviation metric” is a numerical, descriptive, or categorical indication that indicates how a first item differentiates from a second item. Deviation metric may include a numerical/percentile score such as 8/10, 80/100, or 80%. Deviation metric may include a classification such as “very healthy”, “healthy”, “unhealthy”, or “very unhealthy”. Deviation metric may include a descriptor such as “no action needed” or “requiring immediate intervention”.

With continued reference to FIG. 1, assessment and/or self-assessment data, and/or automated or other assessment results, may be obtained from a third-party device 116. Third-party device 116 may include, without limitation, a server or other device (not shown) that performs automated cognitive, psychological, behavioral, personality, or other assessments. Third-party device 116 may include a device operated by an informed advisor. An informed advisor may include any medical professional who may assist and/or participate in the medical treatment of a user. An informed advisor may include a medical doctor, nurse, physician assistant, pharmacist, yoga instructor, nutritionist, spiritual healer, meditation teacher, fitness coach, health coach, life coach, and the like.

With continued reference to FIG. 1, physiological data may include data describing one or more test results, including results of mobility tests, stress tests, dexterity tests, endocrinal tests, genetic tests, and/or electromyographic tests, biopsies, radiological tests, genetic tests, and/or sensory tests. Persons skilled in the art, upon reviewing the entirety of this disclosure, will be aware of various additional examples of at least a physiological sample consistent with this disclosure.

With continued reference to FIG. 1, physiological data may include one or more user body measurements. A “user body measurement” as used in this disclosure, includes a measurable indicator of the severity, absence, and/or presence of a disease state. A “disease state” as used in this disclosure, includes any harmful deviation from the normal structural and/or function state of a human being. A disease state may include any medical condition and may be associated with specific symptoms and signs. A disease state may be classified into different types including infectious diseases, deficiency diseases, hereditary diseases, and/or physiological diseases. For instance and without limitation, internal dysfunction of the immune system May produce a variety of different diseases including immunodeficiency, hypersensitivity, allergies, and/or autoimmune disorders.

With continued reference to FIG. 1, user body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance, and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

With continued reference to FIG. 1, epigenetic, as used herein, includes any user body measurements describing changes to a genome that do not involve corresponding changes in nucleotide sequence. Epigenetic body measurement may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic body measurements may include data describing one or more states of methylation of genetic material.

With continued reference to FIG. 1, gut-wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 1, gut-wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut-wall includes muscular layer which contains smooth muscle that aids in peristalsis and the movement of digested material out of and along the gut. Gut-wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 1, gut-wall body measurement may include data describing one or more test results including results of gut-wall function, gut-wall integrity, gut-wall strength, gut-wall absorption, gut-wall permeability, intestinal absorption, gut-wall barrier function, gut-wall absorption of bacteria, gut-wall malabsorption, gut-wall gastrointestinal imbalances and the like.

With continued reference to FIG. 1, gut-wall body measurement may include any data describing blood test results of creatinine levels, lactulose levels, zonulin levels, and mannitol levels. Gut-wall body measurement may include blood test results of specific gut-wall body measurements including d-lactate, endotoxin lipopolysaccharide (LPS) Gut-wall body measurement may include data breath tests measuring lactulose, hydrogen, methane, lactose, and the like. Gut-wall body measurement may include blood test results describing blood chemistry levels of albumin, bilirubin, complete blood count, electrolytes, minerals, sodium, potassium, calcium, glucose, blood clotting factors,

With continued reference to FIG. 1, gut-wall body measurement may include one or more stool test results describing presence or absence of parasites, firmicutes, Bacteroidetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 1, gut-wall body measurement may include one or more stool test results describing presence, absence, and/or measurement of microorganisms including bacteria, archaea, fungi, protozoa, algae, viruses, parasites, worms, and the like. Stool test results may contain species such as Bifidobacterium species, campylobacter species, Clostridium difficile, cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 1, gut-wall body measurement may include one or more microscopic ova exam results, microscopic parasite exam results, protozoan polymerase chain reaction test results and the like. Gut-wall body measurement may include enzyme-linked immunosorbent assay (ELISA) test results describing immunoglobulin G (IgG) food antibody results, immunoglobulin E (IgE) food antibody results, Ig E mold results, IgG spice and herb results. Gut-wall body measurement may include measurements of calprotectin, eosinophil protein x (EPX), stool weight, pancreatic elastase, total urine volume, blood creatinine levels, blood lactulose levels, blood mannitol levels.

With continued reference to FIG. 1, gut-wall body measurement may include one or more elements of data describing one or more procedures examining gut including for example colonoscopy, endoscopy, large and small molecule challenge and subsequent urinary recovery using large molecules such as lactulose, polyethylene glycol-3350, and small molecules such as mannitol, L-rhamnose, polyethyleneglycol-400. Gut-wall body measurement may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 1, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithies' and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, microbiome body measurement may include one or more stool test results describing presence, absence, and/or measurement of microorganisms including bacteria, archaea, fungi, protozoa, algae, viruses, parasites, worms, and the like. Stool test results may contain species such as Ackerman's muciniphila, Anaerotruncus colihominis, bacteriology, Bacteroides vulgates', Bacteroides-Prevotella, Barnesiella species, Bifidobacterium longarm, Bifidobacterium species, Butyrivbrio crossotus, Clostridium species, Collinsella acrofaciens, fecal color, fecal consistency, Coprococcus cutactus, Desulfovibrio piger, Escherichia coli, Faccalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 1, microbiome body measurement may include one or more stool tests results that identify all microorganisms living a user's gut including bacteria, viruses, archaea, yeast, fungi, parasites, and bacteriophages. Microbiome body measurement may include DNA and RNA sequences from live microorganisms that may impact a user's health. Microbiome body measurement may include high resolution of both species and strains of all microorganisms. Microbiome body measurement may include data describing current microbe activity. Microbiome body measurement may include expression of levels of active microbial gene functions. Microbiome body measurement may include descriptions of sources of disease-causing microorganisms, such as viruses found in the gastrointestinal tract such as raspberry bushy swarf virus from consuming contaminated raspberries or Pepino mosaic virus from consuming contaminated tomatoes.

With continued reference to FIG. 1, microbiome body measurement may include one or more blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 1, microbiome body measurement may include one or more breath test results that identify certain strains of microorganisms that may be present in certain areas of a user's body. This may include for example, lactose intolerance breath tests, methane-based breath tests, hydrogen-based breath tests, fructose-based breath tests, Helicobacter pylori breath test, fructose intolerance breath test, bacterial overgrowth syndrome breath tests and the like.

With continued reference to FIG. 1, microbiome body measurement may include one or more urinary analysis results for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

With continued reference to FIG. 1, nutrient as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron.

With continued reference to FIG. 1, nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 1, nutrient body measurement may include one or more blood test results that identify extracellular and intracellular levels of nutrients. Nutrient body measurement may include blood test results that identify serum, white blood cell, and red blood cell levels of nutrients. For example, nutrient body measurement may include serum, white blood cell, and red blood cell levels of micronutrients such as Vitamin A, Vitamin B1, Vitamin B2, Vitamin B3, Vitamin B6, Vitamin B12, Vitamin B5, Vitamin C, Vitamin D, Vitamin E, Vitamin K1, Vitamin K2, and folate.

With continued reference to FIG. 1, nutrient body measurement may include one or more blood test results that identify serum, white blood cell and red blood cell levels of nutrients such as calcium, manganese, zinc, copper, chromium, iron, magnesium, copper to zinc ratio, choline, inositol, carnitine, methylmalonic acid (MMA), sodium, potassium, asparagine, glutamine, serine, coenzyme q10, cysteine, alpha lipoic acid, glutathione, selenium, cicosapentaenoic acid (EPA), docosahexaenoic acid (DHA), docosapentaenoic acid (DPA), total omega-3, lauric acid, arachidonic acid, oleic acid, total omega 6, and omega 3 index.

With continued reference to FIG. 1, nutrient body measurement may include one or more salivary test results that identify levels of nutrients including any of the nutrients as described herein. Nutrient body measurement may include hair analysis of levels of nutrients including any of the nutrients as described herein.

With continued reference to FIG. 1, genetic as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 1, genetic body measurement may include one or more results from one or more blood tests, hair tests, skin tests, urine, amniotic fluid, buccal swabs and/or tissue test to identify a user's particular sequence of nucleotides, genes, chromosomes, and/or proteins. Genetic body measurement may include tests that example genetic changes that may lead to genetic disorders. Genetic body measurement may detect genetic changes such as deletion of genetic material or pieces of chromosomes that may cause Duchenne Muscular Dystrophy. Genetic body measurement may detect genetic changes such as insertion of genetic material into DNA or a gene such as the BRCA1 gene that is associated with an increased risk of breast and ovarian cancer due to insertion of 2 extra nucleotides. Genetic body measurement may include a genetic change such as a genetic substitution from a piece of genetic material that replaces another as seen with sickle cell anemia where one nucleotide is substituted for another. Genetic body measurement may detect a genetic change such as a duplication when extra genetic material is duplicated one or more times within a person's genome such as with Charcot-Marie Tooth disease type 1. Genetic body measurement may include a genetic change such as an amplification when there is more than a normal number of copies of a gene in a cell such as HER2 amplification in cancer cells. Genetic body measurement may include a genetic change such as a chromosomal translocation when pieces of chromosomes break off and reattach to another chromosome such as with the BCR-ABL1 gene sequence that is formed when pieces of chromosome 9 and chromosome 22 break off and switch places. Genetic body measurement may include a genetic change such as an inversion when one chromosome experiences two breaks and the middle piece is flipped or inverted before reattaching. Genetic body measurement may include a repeat such as when regions of DNA contain a sequence of nucleotides that repeat a number of times such as for example in Huntington's disease or Fragile X syndrome. Genetic body measurement may include a genetic change such as a trisomy when there are three chromosomes instead of the usual pair as seen with Down syndrome with a trisomy of chromosome 21, Edwards syndrome with a trisomy at chromosome 18 or Patau syndrome with a trisomy at chromosome 13. Genetic body measurement may include a genetic change such as monosomy such as when there is an absence of a chromosome instead of a pair, such as in Turner syndrome.

With continued reference to FIG. 1, genetic body measurement may include an analysis of COMT gene that is responsible for producing enzymes that metabolize neurotransmitters. Genetic body measurement may include an analysis of DRD2 gene that produces dopamine receptors in the brain. Genetic body measurement may include an analysis of ADRA2B gene that produces receptors for noradrenaline. Genetic body measurement may include an analysis of 5-HTTLPR gene that produces receptors for serotonin. Genetic body measurement may include an analysis of BDNF gene that produces brain derived neurotrophic factor. Genetic body measurement may include an analysis of 9p21 gene that is associated with cardiovascular disease risk. Genetic body measurement may include an analysis of APOE gene that is involved in the transportation of blood lipids such as cholesterol. Genetic body measurement may include an analysis of NOS3 gene that is involved in producing enzymes involved in regulating vasodilation and vasoconstriction of blood vessels.

With continued reference to FIG. 1, genetic body measurement may include ACE gene that is involved in producing enzymes that regulate blood pressure. Genetic body measurement may include SLCO1B1 gene that directs pharmaceutical compounds such as statins into cells. Genetic body measurement may include FUT2 gene that produces enzymes that aid in absorption of Vitamin B12 from digestive tract. Genetic body measurement may include MTHFR gene that is responsible for producing enzymes that aid in metabolism and utilization of Vitamin B9 or folate. Genetic body measurement may include SHMT1 gene that aids in production and utilization of Vitamin B9 or folate. Genetic body measurement may include MTRR gene that produces enzymes that aid in metabolism and utilization of Vitamin B12. Genetic body measurement may include MTR gene that produces enzymes that aid in metabolism and utilization of Vitamin B12. Genetic body measurement may include FTO gene that aids in feelings of satiety or fulness after eating. Genetic body measurement may include MC4R gene that aids in producing hunger cues and hunger triggers. Genetic body measurement may include APOA2 gene that directs body to produce ApoA2 thereby affecting absorption of saturated fats. Genetic body measurement may include UCP1 gene that aids in controlling metabolic rate and thermoregulation of body. Genetic body measurement may include TCF7L2 gene that regulates insulin secretion. Genetic body measurement may include AMY1 gene that aids in digestion of starchy foods. Genetic body measurement may include MCM6 gene that controls production of lactase enzyme that aids in digesting lactose found in dairy products. Genetic body measurement may include BCMO1 gene that aids in producing enzymes that aid in metabolism and activation of Vitamin A. Genetic body measurement may include SLC23A1 gene that produce and transport Vitamin C. Genetic body measurement may include CYP2R1 gene that produce enzymes involved in production and activation of Vitamin D. Genetic body measurement may include GC gene that produce and transport Vitamin D. Genetic body measurement may include CYPIA2 gene that aid in metabolism and elimination of caffeine. Genetic body measurement may include CYP17A1 gene that produce enzymes that convert progesterone into androgens such as androstenedione, androstendiol, dehydroepiandrosterone, and testosterone.

With continued reference to FIG. 1, genetic body measurement may include CYP19A1 gene that produce enzymes that convert androgens such as androstenedione and testosterone into estrogens including estradiol and estrone. Genetic body measurement may include SRD5A2 gene that aids in production of enzymes that convert testosterone into dihydrotestosterone. Genetic body measurement may include UFT2B17 gene that produces enzymes that metabolize testosterone and dihydrotestosterone. Genetic body measurement may include CYP1A1 gene that produces enzymes that metabolize estrogens into 2 hydroxy-estrogen. Genetic body measurement may include CYP1B1 gene that produces enzymes that metabolize estrogens into 4 hydroxy-estrogen. Genetic body measurement may include CYP3A4 gene that produces enzymes that metabolize estrogen into 16 hydroxy-estrogen. Genetic body measurement may include COMT gene that produces enzymes that metabolize 2 hydroxy-estrogen and 4 hydroxy-estrogen into methoxy estrogen. Genetic body measurement may include GSTT1 gene that produces enzymes that eliminate toxic by-products generated from metabolism of estrogens. Genetic body measurement may include GSTM1 gene that produces enzymes responsible for eliminating harmful by-products generated from metabolism of estrogens. Genetic body measurement may include GSTPI gene that produces enzymes that eliminate harmful by-products generated from metabolism of estrogens. Genetic body measurement May include SOD2 gene that produces enzymes that eliminate oxidant by-products generated from metabolism of estrogens.

With continued reference to FIG. 1, metabolic, as used herein, includes any process that converts food and nutrition into energy. Metabolic may include biochemical processes that occur within the body. Metabolic body measurement may include blood tests, hair tests, skin tests, amniotic fluid, buccal swabs and/or tissue test to identify a user's metabolism. Metabolic body measurement may include blood tests that examine glucose levels, electrolytes, fluid balance, kidney function, and liver function. Metabolic body measurement may include blood tests that examine calcium levels, albumin, total protein, chloride levels, sodium levels, potassium levels, carbon dioxide levels, bicarbonate levels, blood urea nitrogen, creatinine, alkaline phosphatase, alanine amino transferase, aspartate amino transferase, bilirubin, and the like.

With continued reference to FIG. 1, metabolic body measurement may include one or more blood, saliva, hair, urine, skin, and/or buccal swabs that examine levels of hormones within the body such as 11-hydroxy-androstercone, 11-hydroxy-etiocholanolone, 11-keto-androsterone, 11-keto-etiocholanolone, 16 alpha-hydroxyestrone, 2-hydroxyestrone, 4-hydroxyestrone, 4-methoxyestrone, androstanediol, androsterone, creatinine, DHEA, estradiol, estriol, estrone, ctiocholanolone, pregnanediol, pregnanestriol, specific gravity, testosterone, tetrahydrocortisol, tetrahydrocrotisone, tetrahydrodeoxycortisol, allo-tetrahydrocortisol.

With continued reference to FIG. 1, metabolic body measurement may include one or more metabolic rate test results such as breath tests that may analyze a user's resting metabolic rate or number of calories that a user's body burns each day rest. Metabolic body measurement may include one or more vital signs including blood pressure, breathing rate, pulse rate, temperature, and the like. Metabolic body measurement may include blood tests such as a lipid panel such as low density lipoprotein (LDL), high density lipoprotein (HDL), triglycerides, total cholesterol, ratios of lipid levels such as total cholesterol to HDL ratio, insulin sensitivity test, fasting glucose test, Hemoglobin AIC test, adipokines such as leptin and adiponectin, neuropeptides such as ghrelin, pro-inflammatory cytokines such as interleukin 6 or tumor necrosis factor alpha, anti-inflammatory cytokines such as interleukin 10, markers of antioxidant status such as oxidized low-density lipoprotein, uric acid, paraoxonase 1. Persons skilled in the art, upon reviewing the entirety of this disclosure, will be aware of various additional examples of physiological data that may be used consistently with descriptions of systems and methods as provided in this disclosure.

With continued reference to FIG. 1, physiological data may be obtained from a physically extracted sample. A “physical sample” as used in this example, may include any sample obtained from a human body of a user. A physical sample may be obtained from a bodily fluid and/or tissue analysis such as a blood sample, tissue, sample, buccal swab, mucous sample, stool sample, hair sample, fingernail sample and the like. A physical sample may be obtained from a device in contact with a human body of a user such as a microchip embedded in a user's skin, a sensor in contact with a user's skin, a sensor located on a user's tooth, and the like. Physiological data may be obtained from a physically extracted sample. A physical sample may include a signal from a sensor configured to detect physiological data of a user and record physiological data as a function of the signal. A sensor may include any medical sensor and/or medical device configured to capture sensor data concerning a patient, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmographic equipment, or the like. A sensor may include any electromagnetic sensor, including without limitation electroencephalographic sensors, magnetoencephalographic sensors, electrocardiogramansors, electromyographic sensors, or the like. A sensor may include a temperature sensor. A sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. At least a wearable and/or mobile device sensor may detect heart rate or the like. A sensor may detect any hematological parameter including blood oxygen level, pulse rate, heart rate, pulse rhythm, blood sugar, and/or blood pressure. A sensor may be configured to detect internal and/or external biomarkers and/or readings. A sensor may be a part of system 100 or may be a separate device in communication with system 100. User data may include a profile, such as a psychological profile, generated using previous item selections by the user; profile may include, without limitation, a set of actions and/or navigational actions performed as described in further detail below, which may be combined with biological extraction 108 data and/or other user data for processes such as classification to user sets as described in further detail below.

Still referring to FIG. 1, retrieval of biological extraction 108 may include, without limitation, reception of biological extraction 108 from another computing device 104 such as a device operated by a medical and/or diagnostic professional and/or entity, a user client device, and/or any device suitable for use as a third-party device as described in further detail below. Biological extraction 108 may be received via a questionnaire posted and/or displayed on a third-party device as described below, inputs to which may be processed as described in further detail below. Alternatively or additionally, biological extraction 108 may be stored in and/or retrieved from a user database 112. User database 112 may include any data structure for ordered storage and retrieval of data, which may be implemented as a hardware or software module. A user database 112 may be implemented, without limitation, as a relational database, a key-value retrieval datastore such as a NOSQL database, or any other format or structure for use as a datastore that a person skilled in the art would recognize as suitable upon review of the entirety of this disclosure. A user database 112 may include a plurality of data entries and/or records corresponding to user tests as described above. Data entries in a user database 112 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a user database 112 may reflect categories, cohorts, and/or populations of data consistently with this disclosure. User database 112 may be located in memory of computing device 104 and/or on another device in and/or in communication with system 100.

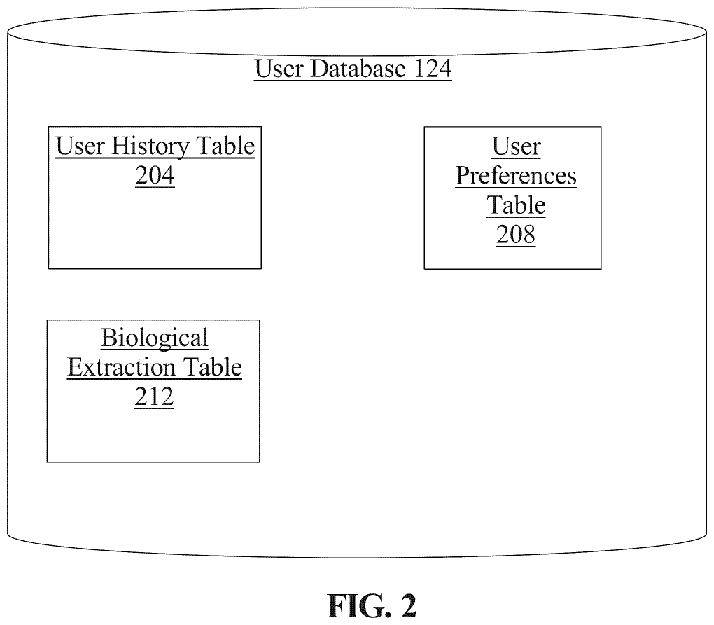

Referring now to FIG. 2, an exemplary embodiment of a user database 112 is illustrated. One or more tables in user database 112 may include, without limitation, a user history table 204, which may be used to store data describing past user requests, educational activities, employment history, or the like. One or more tables in user database 112 may include, without limitation, user preference table 208, which may be used to store one or more user preferences with regard to cost, geographic location, or the like. One or more tables in user database 112 may include, without limitation, a biological extraction table 212, which may be used to store biological extraction 108 data. Persons skilled in the art, upon reviewing the entirety of this disclosure, will be aware of various alternative or additional data which may be stored in user database 112, including without limitation any data concerning any user activity, demographics, profile information, viewing and/or media consumption history, or the like.

Referring again to FIG. 1, and as noted above, retrieval of biological extract may be performed multiple sequential and/or concurrent times, and any process using biological extract as described below may be performed multiple sequential and/or concurrent times; likewise, biological extract may include multiple elements of physiological data, which may be used in combination for any determination and/or other processes as described below.

Still referring to FIG. 1, computing device 104 is configured to receive an educational inquiry from a third-party device 116. An “educational inquiry,” as used herein, is a datum requesting a recommended choice of educational resources. An educational inquiry may include, without limitation, an inquiry seeking to determine what school or schools may be best for a user to attend, where a school may include grade school, middle school, high school, college, secondary education and the like. An educational inquiry may seek a recommendation of adult education classes a user should enroll in. An educational inquiry may seek a determination of what teaching style is best for a user to learn information. An educational inquiry may seek a determination of what learning style is best for a user to understand information. An educational inquiry may seek to determine an appropriate class size for a user. An educational inquiry may seek to determine which subject of study, degree, major, concentration, or the like is appropriate for a user. An educational inquiry may include an inquiry regarding a suitable educational institution. An educational inquiry may include an inquiry regarding a suitable form of instruction, such as large class, lecture hall, live-streamed, and/or online instruction. An educational inquiry may include an inquiry regarding a learning style of the user. An educational inquiry may include an inquiry regarding a program of study, such as a 4-year program versus 2-year program, a graduate program, a program offering internships, coops, or the like. An educational inquiry may include an inquiry regarding mental health supports, protocols, and/or procedures at an institution and/or program. An educational inquiry may include an inquiry regarding special needs supports, protocols, and/or procedures at an institution and/or program. An educational inquiry may include an inquiry regarding disability supports, protocols, and/or procedures, such as accommodations for learning, mental, and/or physical disabilities, at an institution and/or program. An educational inquiry may include a nutrition-related educational inquiry. Nutrition-related educational inquiry may include questions pertaining to one or more types of food or drink to start or stop consuming, one or more changes or adjustments in dietary habits, one or more suspected cases of suspected nutrient deficiency, among others, consistent with details described in this disclosure. An educational inquiry may include at least in intended outcome, such as weight loss, reduced hypertension, a reduced cholesterol level, a reduced stress level, an improvement in depressive disorders, or any similar metric that may signify an improved well-being of an individual. Such intended outcome may be self-reported or generated by system 100 based on one or more elements of educational inquiry. Similarly, an educational inquiry may include one or more symptoms that an individual is looking to improve, consistent with details described above. As nonlimiting examples, a user may indicate “I want to sleep better”, “I want to have more energy when I wake up”, or “I want to reduce my body fat level to below 20%”. Persons skilled in the art, upon reviewing the entirety of this disclosure, will be aware of various additional examples of educational inquiries that are within the scope of this disclosure. Each inquiry and/or type of inquiry may correspond to one or more machine-learning processes 120, as described below, which may be used to generate responses as described below.

With continued reference to FIG. 1, third-party device 116 may include any device suitable for use as computing device 104, as described above. Third-party device 116 may include, without limitation, a device operated by user, a device operated by an educational institution, a device operated by an entity and/or person offering educational advice and/or assistance in answering educational inquiries, such as a purveyor of a website offering such a service; alternatively or additionally, computing device 104 may offer such a service via a graphical user interface such as a web page or native application on a user-operated computing device 104, which may be third-party device 116. Educational inquiry may be generated by third-party device 116 automatically, assembled using user selections of menu items, for instance from drop-down menus, user selections of checkboxes, radio buttons, or other pre-populated and/or pre-arranged data elements that may be selected via user actions.

Alternatively, or additionally, and further referring to FIG. 1, educational request may include a textual word and/or phrase, such as an educational request that a person might enter on a graphical user interface as described above. Computing device 104 may parse a textual word or phrase to generate one or more keywords, where keywords may include single words and/or phrases of two or more words. Computing device 104 may, for instance, tokenize a textual word or phrase to separate the textual word or phrase into individual words. Computing device 104 may filter out “stop words” that do not convey meaning, such as “of,” “a,” “an,” “the,” or the like. Computing device 104 may use words parsed from educational request directly as keywords for retrieval from a database, index, or other datastore. Alternatively, or additionally, Computing device 104 may generate phrases to use as keywords and/or map one or more words or phrases from a textual word or phrase to a keyword query for retrieval from a database, index, or other datastore using a language processing module. Language processing module may include any hardware and/or software module. Language processing module may be configured to extract, from the one or more documents, one or more words. One or more words may include, without limitation, strings of one or characters, including without limitation any sequence or sequences of letters, numbers, punctuation, diacritic marks, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, a language processing module may operate to produce a language processing model. Language processing model may include a program automatically generated by computing device 104 and/or language processing module to produce associations between one or more words extracted from at least a document and detect associations, including without limitation mathematical associations, between such words. Associations between language elements, where language elements include for purposes herein extracted words, may include, without limitation, mathematical associations, including without limitation statistical correlations between any language element and any other language element and/or language elements. Statistical correlations and/or mathematical associations may include probabilistic formulas or relationships.

With continued reference to FIG. 1, language processing module and/or computing device 104 may generate the language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between words. There may be a finite number of other words and/or phrases to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation eosine similarity, which measures the similarity of two vectors by evaluating the eosine of the angle between the vectors, which can be computed using a dot product of the two vectors divided by the lengths of the two vectors. Degree of similarity may include any other geometric measure of distance between vectors.

Still referring to FIG. 1, language processing module may use a corpus of documents to generate associations between language elements in a language processing module, and language processing module and/or computing device 104 may then use such associations to analyze words extracted from one or more documents and determine relationships between such words. Corpus of documents may include any set of documents, such as a plurality of web pages, textual conversation logs, articles, blog posts, excerpts from and/or electronic texts of books or the like. Alternatively, or additionally, language processing module may generate a model using correlations between words and/or phrases compiled by a third party, such as the n-grams database provided by Alphabet, Inc. of Mountain View, CA.

With continued reference to FIG. 1, a textual word or phrase entry may be performed by offering an entry field to a user in a graphical user interface (GUI), which may be provided to a display of a user device. GUI may offer user options such as date ranges, content categories such as videos, images, general content, news, shopping, maps, and the like. GUI may provide one or more options for forms of search; for instance, GUI may provide natural language searching, which may utilize a language processing module as described above to process sentences or phrases entered by a user in a manner similar to a conversational question. GUI may provide an option to enter a Boolean search; in other words, computing device 104 may parse a textual word or phrase providing educational request for Boolean operators such as AND, OR, NOT and the like, and apply logic of such operators to search results using program commands implementing Boolean logic. GUI may be used to offer proximity search, where a distance between keywords from query in content at result URLs is used as a ranking or selection criterion thereof; alternatively or additionally, searching for an exact phrase and/or synonymous or closely related phrases may be performed using, without limitation, language processing modules and/or language processing models such as vector spaces, effectively adding proximity between keywords and/or synonyms thereof to criteria for finding and/or ranking search results.

Still referring to FIG. 1, computing device 104 is configured to select at least a machine-learning process 120, based on the educational inquiry. A “machine learning process” is a process that automatedly uses a body of data known as “training data” and/or a “training set” to generate an algorithm that will be performed by a computing device 104/module to produce outputs given data provided as inputs; this is in contrast to a non-machine learning software program where the commands to be executed are determined in advance by a user and written in a programming language. At least a machine-learning process 120 may be used by computing device 104 to generate an inquiry response as described in further detail below.

Still referring to FIG. 1, computing device 104 may store at least a machine-learning process 120 in and/or select at least a machine-learning process 120 from a process database 124. Process database 124 may include any database suitable for use as a user database 112 as described above; process database 124 may, as a non-limiting example, relate each machine-learning process 120 to an entry in one or more indices, such as indices of machine-learning process 120 identifiers, indices and/or links to tables of user data, biological extraction 108 data, and/or identifiers of one or more educational inquiries. For instance, and without limitation, one or more user selections of graphical user interface elements as described above, such as without limitation drop-down menu items or the like, may be combined to query process database 124, returning at least a machine-learning process 120. One or more user textual entries may be mapped by a language processing module, model, and/or process to data used as index entries, which may then be used to form a query, which may combine such entries with user selections, to retrieve at least a machine-learning process 120.

Alternatively or additionally, and still referring to FIG. 1, computing device 104 may select at least a machine-learning process 120 using machine learning. For instance, and without limitation, computing device 104 may be configured to select the at least a machine-learning process 120 using a process classifier 128 that inputs an educational inquiry and outputs at least a machine-learning process 120. A “classifier,” as used in this disclosure, is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. Process classifier 128 may include a classifier configured to input user data and output user set identifiers; process classifier 128 may include a classifier configured to input biological extractions 108 and output user set identifiers.

Computing device 104 and/or another device may generate process classifier 128 using a classification algorithm, defined as a processes whereby a computing device 104 derives a classifier from process classification training data 132. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

With continued reference to FIG. 1, process classification training data 132 may be any training data. “Training data,” as used in this disclosure, is data containing correlations that a machine-learning process 120 may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes 120 as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by computing device 104 may correlate any input data as described in this disclosure to any output data as described in this disclosure.

Still referring to FIG. 1, training data used to generate process classifier 128 may include, without limitation, a plurality of educational inquiries, each educational inquiry including one or more educational inquiries, and one or more correlated identifiers of machine-learning processes 120. Such correlations may be identified, without limitation, by user entries and/or expert entries identifying associations between particular selections and/or text entries and particular machine-learning processes 120 and/or by user feedback; for instance, an earlier iteration of a method as disclosed herein may generate an outcome and receive user feedback indicating a degree of accuracy of the outcome and/or appropriateness of a category of question used to produce the outcome. Process classification training data 132 may additionally include entries correlating biological extractions 108 to identifiers of machine-learning processes 120, which correlations may be identified as described above.

Still referring to FIG. 1, computing device 104 may be configured to generate process classifier 128 using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate process classifier 128 using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as eosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=Σᵢ₌₀ⁿaᵢ², where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values. As a non-limiting example, K-nearest neighbors algorithm may be configured to classify an input vector including a plurality of user-entered words and/or phrases, user selections, and/or elements of physiological data to clusters representing machine-learning processes 120.

Still referring to FIG. 1, computing device 104 may additionally be configured to select at least a machine-learning process 120 as a function of biological extraction 108. For instance, a classifier may be generated, for instance using classification algorithms and/or training data as described above, that inputs biological extraction 108 and outputs at least a machine-learning process 120. This may be based, without limitation, on one or more identified mental, emotional, psychological, and/or physiological needs of user as identified by and/or in biological extraction 108 such needs may include, without limitation, diagnoses, disabilities, or other conditions affecting learning ability, such as learning disabilities, autism spectrum disorders, and dyslexia. Additionally, and/or alternatively, such needs may include or reflect a nutritional need, such as one or more nutrients that a person is lacking, a special diet. Nutrient may include any type of nutrient disclosed in this disclosure. Special diet may include a ketogenic diet, characterized by high fat and low carbohydrate intake; a paleo diet, which focuses on foods presumed to be available to Paleolithic humans; a Mediterranean diet, which emphasizes fruits, vegetables, whole grains, and healthy fats; a vegan diet, which excludes all animal products; a vegetarian diet, which eliminates meat but may include dairy and eggs; a pescatarian diet, which includes fish but excludes other meats; a gluten-free diet, which avoids all forms of gluten; a low-Fermentable Oligosaccharides, Disaccharides, Monosaccharides, and Polyols (FODMAP) diet, which is designed to alleviate symptoms of irritable bowel syndrome (IBS) by reducing certain fermentable carbs; a Dietary Approaches to Stop Hypertension (DASH) diet, which aims to lower blood pressure through reduced sodium intake and a balanced intake of nutrients; a low-carb diet, which limits carbohydrate intake to manage weight and blood sugar levels; an intermittent fasting diet, which alternates periods of eating with fasting to aid in weight loss and metabolic health; a whole 30 diet, which eliminates sugar, alcohol, grains, legumes, soy, and dairy for 30 days to reset the body's metabolism; an anti-inflammatory diet, which includes foods that reduce inflammation, such as fatty fish and leafy greens; an alkaline diet, which focuses on foods that affect the pH balance of the body, promoting fruits, vegetables, nuts, and seeds; a carnivore diet, which consists solely of animal products, in contrast to plant-based diets; an elimination diet, which identifies food intolerances by removing and gradually reintroducing potential allergens; an SCD (Specific Carbohydrate Diet), which restricts certain carbohydrates to manage conditions like Crohn's disease and ulcerative colitis; a raw food diet, which emphasizes uncooked, unprocessed foods. Identification of needs may be performed, without limitation by generation of one or more prognostic labels, for instance as described in U.S. Nonprovisional application Ser. No. 16/372,512, filed on Apr. 2, 2019, and titled “METHODS AND SYSTEMS FOR UTILIZING DIAGNOSTICS FOR INFORMED VIBRANT CONSTITUTIONAL GUIDANCE,” the entirety of which is incorporated herein by reference. Identification of needs may be performed using a user effective age, which may be calculated as described in U.S. Nonprovisional application Ser. No. 16/558,502, filed on Sep. 3, 2019, and entitled “SYSTEMS AND METHODS FOR SELECTING AN INTERVENTION BASED ON EFFECTIVE AGE,” the entirety of which is incorporated herein by reference. In an embodiment, computing device 104 may generate a first set of one or more machine-learning processes 120 as a function of educational inquiry, generate a second set of one or more machine-learning processes 120 as a function of biological extraction 108, and combine the first set and the second set as the at least a machine-learning process 120; combination may include detecting that two of the selected machine-learning processes 120 are duplicates and eliminating one of the two selected machine-learning processes 120. Alternatively or additionally, process classifier 128 may take combinations of physiological data and educational inquiry data as inputs and output machine-learning processes 120. Selection of at least a machine-learning process 120 may alternatively or additionally be performed using any machine-learning process 120 described in this disclosure. Alternatively or additionally, user may one or more criteria according to which user wishes to evaluate and/or rank options, which computing device 104 may use to select at least a machine-learning process 120, for instance by retrieval from process database 124 and/or using process classifier 128.

With continued reference to FIG. 1, computing device 104 is configured to generate, using at least a machine-learning process 120 and biological extraction 108, an inquiry response 136. An “inquiry response,” as used in this disclosure, is an element of data containing a recommended educational decision, such as a recommended educational decision that answers a question and/or request for guidance in an educational inquiry. As a non-limiting example, an educational inquiry that contains a question regarding what a user should major in at college may cause computing device 104 to generate, using at least a machine-learning process 120 a response that contains a ranked list of majors the user should consider majoring in, an educational inquiry that contains a question regarding what school a user should attend may cause computing device 104 to generate, using at least a machine-learning process 120 a response that lists a recommended school and/or a ranked list of schools. As a further non-limiting example, an educational inquiry that contains a question regarding what style of instruction a user should receive may cause computing device 104 to generate, using at least a machine-learning process 120, a response that recommends an instructional style and/or a ranked list of instructional styles. In some cases, an inquiry response may include nutritional support resources such as one or more planned meals, one or more certified vendors that may provide such planned meals, one or more recommended courses to consider enrolling in, one or more recommended articles or blogs to read, one or more healthy recipes to try out, one or more food items to focus on or avoid, and the like. In some cases, an inquiry response may include one or more recommended behavioral changes, such as a reduced number of times of going to restaurants per week, a more frequent exercise routine, a more active social life that may help one destress, among others. In some cases, inquiry response may be generated as a function of one or more preferred dietary routines pertaining to user, as described above. In some cases, inquiry response may adopt certain pedagogical approaches and/or include certain educational elements, such as one or more explanatory components, to convince user why certain measures or interventions are recommended and the scientific reasoning behind. This may enable user to better understand the potential impacts of these measures or interventions and better motivate the user to achieve their goal. As a nonlimiting example, one or more educational elements may pertain to the importance of habit building. In some cases, inquiry response may include a structured program comprising a plurality of steps. Such steps may begin with less challenging steps that are easier to follow and progressively turn into more challenging steps that require more dedication. Such structured programs may avoid overwhelming users and increase the rate of completion or success. In some cases, educational element may include at least an interactive tool. Interactive tool may include graphics, tabs, or the like to allow user to interact with system 100 using a mouse or a finger tip. Interactive tools may help user track their progress and promote their understanding of educational elements, suggested measures/interventions, such as a recommended meal plan, or the like. In some cases, educational element may include at least a visual aid. Visual aid may be configured to apply visual elements such as animations or color coding to simplify complex concepts and illustrate the benefits of a suggested measure or intervention such as a recommended meal plan. In some cases, inquiry response may be generated as a function of one or more intended outcomes and/or one or more symptoms specified by a user, consistent with details described above.

With continued reference to FIG. 1, in some cases, system 100 may be configured to receive a revised educational inquiry from a user; and update at least a machine-learning model or process 120 and inquiry response 136 as a function of the revised educational inquiry. Updating at least a machine-learning model or process 120 may include adjusting one or more parameters within the machine-learning model or process 120 and/or updating one or more elements of biological extraction training data 140 within the machine-learning model or process 120. In some cases, system 100 may be configured to routinely check in with a user to keep track of the user's progress over an extended period of time and to evaluate whether certain goals or milestones are being met. In some cases, system 100 may be configured to match the at least a nutritional need of the user with at least a nutritional need pertaining to at least a prior user, pair the at least a prior user with the user as a function of the match, and identify the at least a prior user in inquiry response 136 to the user. Accordingly, user may reach out to one or more prior users for advice or inspiration.

Still referring to FIG. 1, each machine-learning process 120 of at least a machine-learning process 120 may be trained using biological extraction training data 140, which may include any training data, as described above, correlating physiological data, prognostic labels, labels identifying treatments, therapies or the like tending to alleviate and/or aid with conditions identified by prognostic labels, and/or other data to each other. Biological extraction training data 140 may be received in any suitable form, including as user entries ranking options according to any or all criteria described above; such user entries may be combined and/or correlated with user biological extraction 108 data. Biological extraction training data 140 may include descriptions of services, facilities, class formats, class sizes, mode of delivery of instruction, mental health support facilities and/or programs, special needs support and/or programs, learning disability support and/or programs, or the like, which may be associated and/or correlated with user entries and/or biological extractions 108 as described above. Biological extraction training data 140 may include expert entries describing programs and/or facilities needed and/or desirable for given elements and/or sets of physiological data; expert entries may be generated and/or received according to any process for reception of expert entries described in this disclosure and/or in any disclosure incorporated herein by reference. Biological extraction training data 140 may include exemplary biological extractions 108 correlated to exemplary nutritional support resources. Biological extraction training data 140 may be dynamically updated as a function of one or more user inputs. Persons skilled in the art, upon reviewing the entirety of this disclosure, will be aware of various alternative or additional entries and/or data usable as biological extraction training data 140 may be provided.

Still referring to FIG. 1, selection of at least a machine-learning process 120 may include selection of a machine-learning model, a training data set to be used in a machine-learning algorithm and/or to produce a machine-learning model, and/or a machine-learning algorithm such as lazy-learning and/or model production, or the like. Computing device 104 may be designed and configured to create a machine-learning model using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 1, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 1, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data.

Continuing to refer to FIG. 1, machine-learning algorithms may include supervised machine-learning algorithms. Supervised machine learning algorithms, as defined herein, include algorithms that receive a training set relating a number of inputs to a number of outputs, and seck to find one or more mathematical relations relating inputs to outputs, where each of the one or more mathematical relations is optimal according to some criterion specified to the algorithm using some scoring function. For instance, a supervised learning algorithm may include elements of physiological data as described above as inputs, inquiry responses 136 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data. Persons skilled in the art, upon reviewing the entirety of this disclosure, will be aware of various possible variations of supervised machine learning algorithms that may be used to determine relation between inputs and outputs. Supervised machine-learning processes 120 may include classification algorithms as defined above.

Still referring to FIG. 1, machine learning processes may include unsupervised processes. An unsupervised machine-learning process 120, as used herein, is a process that derives inferences in datasets without regard to labels; as a result, an unsupervised machine-learning process 120 may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

With continued reference to FIG. 1, machine-learning processes 120 as described in this disclosure may be used to generate machine-learning models. A machine-learning model, as used herein, is a mathematical representation of a relationship between inputs and outputs, as generated using any machine-learning process 120 including without limitation any process as described above, and stored in memory; an input is submitted to a machine-learning model once created, which generates an output based on the relationship that was derived. For instance, and without limitation, a linear regression model, generated using a linear regression algorithm, may compute a linear combination of input data using coefficients derived during machine-learning processes 120 to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 1, at least a machine-learning process 120 may include a lazy-learning process and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data. Heuristic may include selecting some number of highest-ranking associations and/or training data elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

With continued reference to FIG. 1, at least a machine learning process may include a process classifying and/or scoring options according to any criterion used for and/or referred to in any educational inquiry. For instance, and without limitation, at least a machine-learning process 120 may include a mental health suitability classification process and/or scoring algorithm that scores and/or classifies options according to quality and/or availability of mental health supports and/or protocols, as needed by or necessary given biological extraction 108, and/or according to degree of mental health issues in a student body as relevant to biological extraction 108. As a further example, at least a machine-learning process 120 may include a special needs suitability classification process and/or scoring algorithm that scores and/or classifies options according to quality and/or availability of special needs supports and/or protocols, as needed by or necessary given biological extraction 108. As a further example, at least a machine-learning process 120 may include a disability accommodation suitability classification process and/or scoring algorithm that scores and/or classifies options according to quality and/or availability of disability accommodations such as without limitation learning disability accommodations, as needed by or necessary given biological extraction 108.

Continuing to refer to FIG. 1, where at least a machine-learning process 120 is a single process, inquiry response 136 may be presented to user, for instance by presentation of a recommended institution, major, class, instruction style, or learning environment, and/or a ranked list thereof. Where at least a machine-learning process 120 includes a plurality of machine-learning processes 120, computing device 104 may combine outputs thereof to generate educational inquiry. For instance, computing device 104 and/or process classifier 128 as described above may select a first machine-learning process 120 to recommend one or more educational institutions based on suitability of instruction format for a user, a second machine-learning process 120 to recommend one or more educational institutions based on a degree of mental health support, a third machine-learning process 120 to recommend one or more educational institutions based on a degree of support for a given learning disability and/or special need, and/or one or more additional machine-learning processes 120. Each process may produce a recommendation, for instance using a classifier as described above, and/or may produce a score, such as an output of a regression model and/or a degree of proximity in a classifier such as a KNN model and/or lazy-learning process as described above. Scores output by each machine-learning process 120 of at least a machine-learning process 120 may be used in combination to generate a recommendation and/or a ranked list of options. Use of outputs in combination may include calculation of an aggregate score, for instance by adding, multiplying, or averaging scores; options may be ranked by aggregate score and/or an option having a maximal aggregate score may be selected. Individual outputs may alternatively or additionally be presented to a user by category.

Alternatively or additionally, and still referring to FIG. 1, each score corresponding to an option may be used as an attribute of a vector as described above, and proximity of each such vector to a user vector of ideal or optimal values for a user may be calculated using any measure of vector distance as described above; options may be ranked according to degree of proximity to the user vector. User vector may be determined by recording one or more user preferences and quantifying such user preferences as elements of user vector; a user preference may be recorded and quantified per attribute of vectors associated with options. For instance, a graphical user interface may display at user device options to rate one or more priorities absolutely and/or relatively to each other, for instance by providing a numerical rating scale with radio buttons and/or drop-down lists, sliders where a user may set relative importance along a continuum for each user vector attribute, and/or textual entry fields wherein a user may enter numbers reflecting user's personal degree of importance for each field.

With continued reference to FIG. 1, each option of the plurality of options includes an option vector including a plurality of option vector entries; option vector, in any given embodiment, may have entries corresponding to entries in user vector. For instance, and without limitation, each option element may have an associated vector, which may be chosen as a subset of attributes of option element listed in a database, where each attribute indicates an effect each option element has on an attribute in user vector. In an embodiment, selecting and/or ranking one or more options may include generating a loss function of the plurality of options and the user vector, minimizing the loss function, and selecting an option from the plurality of options as a function of minimizing the loss function. A “loss function” as used herein is an expression of an output of which an optimization algorithm minimizes to generate an optimal result. As a non-limiting example, computing device 104 may select an option having an associated vector that minimizes a measure of difference from user vector; measure of difference may include, without limitation, a measure of geometric divergence between option vector and user vector, such as without limitation eosine similarity or may include any suitable error function measuring any degree of divergence and/or aggregation of degrees of divergence, between attributes of user heath quality vector and option vectors. Selection of different loss functions may result in identification of different options as generating minimal outputs. Alternatively or additionally, each of user vector and each option vector may be represented by a mathematical expression having the same form as mathematical expression; computing device 104 may compare the former to the latter using an error function representing average difference between the two mathematical expressions. Error function may, as a non-limiting example, be calculated using the average difference between coefficients corresponding to each variable. An option having a mathematical expression minimizing the error function may be selected, as representing an optimal expression of relative importance of variables to a system or user. In an embodiment, error function and loss function calculations may be combined; for instance, a variable resulting in a minimal aggregate expression of error function and loss function, such as a simple addition, arithmetic mean, or the like of the error function with the loss function, may be selected, corresponding to an option that minimizes total variance from optimal variables while simultaneously minimizing a degree of variance from a set of priorities corresponding to variables. Coefficients of mathematical expression and/or loss function may be scaled and/or normalized; this may permit comparison and/or error function calculation to be performed without skewing by varied absolute quantities of numbers. Server may select a plurality of options to user; for instance, ranking may be maintained of options according to a degree to which they minimize loss function, and a number of highest-ranking options, such as the ten highest ranking options or the like, may be selected. Alternatively or additionally, relative ranking, one or more scores produced by machine-learning processes 120, and/or an aggregate score may be displayed to user along with an option, for instance if the user has selected that option. As a non-limiting example, if a user has selected and/or searched for a particular institution, major, program, or the like, one or more scores and/or rankings associated with the particular institution, major, program, or the like may be displayed to the user.

In an embodiment, and with continued reference to FIG. 1, user vector and vectors associated with options may include one or more attributes generated using at least a machine-learning process 120 and one or more additional attributes; one or more additional attributes may be retrieved and/or received in any suitable manner, including without limitation storage in a database listing such attributes. One or more additional attributes may be provided, for instance, by educational institutions, ranking bodies, third-party information sources, or the like. Such additional attributes may include, without limitation, cost of tuition, housing, materials, or the like, geographic location, academic reputation and/or ranking, degree of difficulty of study in terms of hours per week required or the like, acceptance and/or graduation percentages, programs offered, services offered, and/or any other criterion used to select educational institutions, and/or any other quantifiable attributes usable for and/or suitable for selection of options such as educational institutions, courses of study, or the like as described above. As a non-limiting example, user and option vectors may contain a single output of a single machine-learning process 120, for instance selected based on a user entry, and one or more additional attributes; determination of proximity of option vectors to user vector and selection, scoring, and/or ranking thereby may be performed as described above.

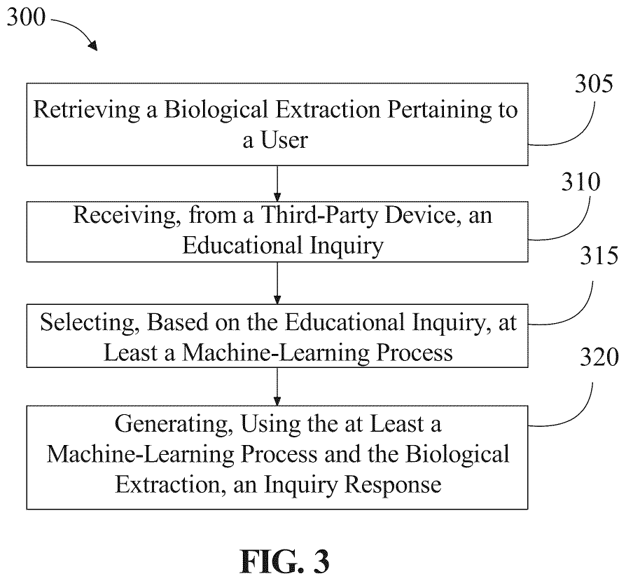

Referring now to FIG. 3, an exemplary embodiment of an artificial intelligence-based method 300 of generating educational inquiry responses 136 from biological extractions 108. At step 305, computing device 104 retrieves a biological extraction 108 pertaining to a user; this may be implemented as described above in reference to FIGS. 1-2. Biological extraction 108 may include a psychological profile; psychological profile may be obtained utilizing a questionnaire performed by the user. Biological extraction 108 may include a genetic sequence.

At step 310, and still referring to FIG. 3, computing device 104 receives an educational inquiry from a third-party device 116; this may be implemented as described above in reference to FIGS. 1-2. Educational inquiry may include an inquiry regarding a suitable educational institution. Educational inquiry may include an inquiry regarding a suitable form of instruction. Educational inquiry may include an inquiry regarding a learning style of the user.

At step 315, and continuing to refer to FIG. 3, the computing device 104 selects at least a machine-learning process 120 based on the educational inquiry; this may be implemented as described above in reference to FIGS. 1-2. Selecting at least a machine-learning process 120 may include selecting the at least a machine-learning process 120 using a classifier that inputs educational inquiry and outputs at least a machine-learning process 120. Selecting the at least a machine-learning process 120 may include selecting the at least a machine-learning process 120 as a function of biological extraction 108. At least a machine-learning process 120 may include a mental health suitability classification process.

At step 320, generating, by the computing device 104 and using the at least a machine-learning process 120 and the biological extraction 108, an inquiry response 136; this may be implemented as described above in reference to FIGS. 1-2.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

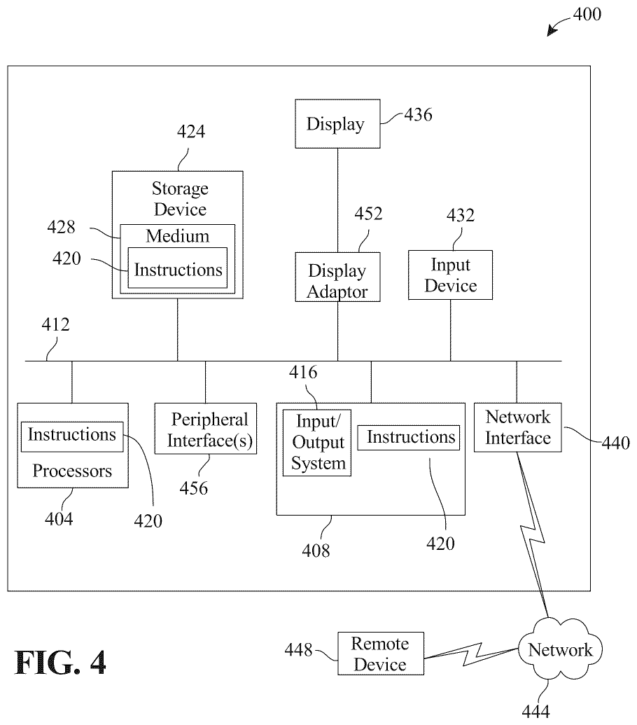

FIG. 4 shows a diagrammatic representation of one embodiment of a computing device in the exemplary form of a computer system 400 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 400 includes a processor 404 and a memory 408 that communicate with each other, and with other components, via a bus 412. Bus 412 may include any of several types of bus structures including, but not limited to, a memory bus, a memory controller, a peripheral bus, a local bus, and any combinations thereof, using any of a variety of bus architectures.

Processor 404 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 404 may be organized according to Von Neumann and/or Harvard architecture as a non-limiting example. Processor 404 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 408 may include various components (e.g., machine-readable media) including, but not limited to, a random-access memory component, a read only component, and any combinations thereof. In one example, a basic input/output system 416 (BIOS), including basic routines that help to transfer information between elements within computer system 400, such as during start-up, may be stored in memory 408. Memory 408 may also include (e.g., stored on one or more machine-readable media) instructions (e.g., software) 420 embodying any one or more of the aspects and/or methodologies of the present disclosure. In another example, memory 408 may further include any number of program modules including, but not limited to, an operating system, one or more application programs, other program modules, program data, and any combinations thereof.

Computer system 400 may also include a storage device 424. Examples of a storage device (e.g., storage device 424) include, but are not limited to, a hard disk drive, a magnetic disk drive, an optical disc drive in combination with an optical medium, a solid-state memory device, and any combinations thereof. Storage device 424 may be connected to bus 412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 424 (or one or more components thereof) may be removably interfaced with computer system 400 (e.g., via an external port connector (not shown)). Particularly, storage device 424 and an associated machine-readable medium 428 may provide nonvolatile and/or volatile storage of machine-readable instructions, data structures, program modules, and/or other data for computer system 400. In one example, software 420 may reside, completely or partially, within machine-readable medium 428. In another example, software 420 may reside, completely or partially, within processor 404.

Computer system 400 may also include an input device 432. In one example, a user of computer system 400 may enter commands and/or other information into computer system 400 via input device 432. Examples of an input device 432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 432 may be interfaced to bus 412 via any of a variety of interfaces (not shown) including, but not limited to, a serial interface, a parallel interface, a game port, a USB interface, a FIREWIRE interface, a direct interface to bus 412, and any combinations thereof. Input device 432 may include a touch screen interface that may be a part of or separate from display device 436, discussed further below. Input device 432 may be utilized as a user selection device for selecting one or more graphical representations in a graphical interface as described above.

A user may also input commands and/or other information to computer system 400 via storage device 424 (e.g., a removable disk drive, a flash drive, etc.) and/or network interface device 440. A network interface device, such as network interface device 440, may be utilized for connecting computer system 400 to one or more of a variety of networks, such as network 444, and one or more remote devices 448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 444, may employ a wired and/or a wireless mode of communication. In general, any network topology may be used. Information (e.g., data, software 420, etc.) may be communicated to and/or from computer system 400 via network interface device 440.

Computer system 400 may further include a video display adapter 452 for communicating a displayable image to a display device, such as display device 436. Examples of a display device include, but are not limited to, a liquid crystal display (LCD), a cathode ray tube (CRT), a plasma display, a light emitting diode (LED) display, and any combinations thereof. Display adapter 452 and display device 436 may be utilized in combination with processor 404 to provide graphical representations of aspects of the present disclosure. In addition to a display device, computer system 400 may include one or more other peripheral output devices including, but not limited to, an audio speaker, a printer, and any combinations thereof. Such peripheral output devices may be connected to bus 412 via a peripheral interface 456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An artificial intelligence system for generating educational inquiry responses from biological extractions, the system comprising a computing device designed and configured to:
retrieve a biological extraction pertaining to a user, wherein the biological extraction comprises nutrition-related physiological data;
identify, based on the biological extraction, at least a nutritional need of the user, wherein the at least a nutritional need of the user is determined using at least an assessment;
receive at least an educational inquiry comprising a nutrition-related educational inquiry;
select, based on the at least an educational inquiry, at least a machine-learning model, wherein selecting the at least a machine-learning model comprises:
receiving biological extraction training data correlating exemplary biological extractions to exemplary nutritional support resources; and
training the at least a machine-learning model using the biological extraction training data; and
generate, using the at least a machine-learning model and the at least a nutritional need of the user, an inquiry response including a plurality of nutritional support resources.

2. The artificial intelligence system of claim 1, wherein the biological extraction further comprises a preferred dietary routine pertaining to the user.

3. The artificial intelligence system of claim 1, wherein computing device is further configured to:
determine a current physical and mental state of the user as a function of the biological extraction and the at least an assessment;
compare the current physical and mental state of the user against an optimum physical and mental state; and
generate a deviation metric as a function of the comparison.

4. The artificial intelligence system of claim 1, wherein the at least an educational inquiry comprises an inquiry regarding at least a suitable educational institution, an inquiry regarding a suitable form of instruction, or an inquiry regarding a learning style of the user.

5. The artificial intelligence system of claim 1, wherein the inquiry response comprises a structured program comprising a plurality of steps.

6. The artificial intelligence system of claim 1, wherein the plurality of nutritional support resources comprises at least an educational element comprising at least an explanatory component.

7. The artificial intelligence system of claim 6, wherein the at least an educational element comprises an element pertaining to an importance of habit building.

8. The artificial intelligence system of claim 6, wherein the at least an educational element comprises at least an interactive tool.

9. The artificial intelligence system of claim 6, wherein the at least an educational element comprises at least a visual aid.

10. The artificial intelligence system of claim 1, wherein generating the inquiry response comprises:
matching the at least a nutritional need of the user with at least a nutritional need pertaining to at least a prior user;
pairing the at least a prior user with the user as a function of the match; and
identifying the at least a prior user in the inquiry response to the user.

11. An artificial intelligence-based method for generating educational inquiry responses from biological extractions, the method comprising:
retrieving a biological extraction pertaining to a user, wherein the biological extraction comprises nutrition-related physiological data;
identifying, based on the biological extraction, at least a nutritional need of the user, wherein the at least a nutritional need of the user is determined using at least an assessment;
receiving at least an educational inquiry comprising a nutrition-related educational inquiry;
selecting, based on the at least an educational inquiry, at least a machine-learning model, wherein selecting the at least a machine-learning model comprises:
receiving biological extraction training data correlating exemplary biological extractions to exemplary nutritional support resources; and
training the at least a machine-learning model using the biological extraction training data; and
generating, using the at least a machine-learning model and the at least a nutritional need of the user, an inquiry response including a plurality of nutritional support resources.

12. The artificial intelligence-based method of claim 11, wherein the biological extraction further comprises a preferred dietary routine pertaining to the user.

13. The artificial intelligence-based method of claim 11, wherein the artificial intelligence-based method further comprises:
determining a current physical and mental state of the user as a function of the biological extraction and the at least an assessment;
comparing the current physical and mental state of the user against an optimum physical and mental state; and
generating a deviation metric as a function of the comparison.

14. The artificial intelligence-based method of claim 11, wherein the at least an educational inquiry comprises an inquiry regarding at least a suitable educational institution, an inquiry regarding a suitable form of instruction, or an inquiry regarding a learning style of the user.

15. The artificial intelligence-based method of claim 11, wherein the inquiry response comprises a structured program comprising a plurality of steps.

16. The artificial intelligence-based method of claim 11, wherein the plurality of nutritional support resources comprises at least an educational element comprising at least an explanatory component.

17. The artificial intelligence-based method of claim 16, wherein the at least an educational element comprises an element pertaining to an importance of habit building.

18. The artificial intelligence-based method of claim 16, wherein the at least an educational element comprises at least an interactive tool.

19. The artificial intelligence-based method of claim 16, wherein the at least an educational element comprises at least a visual aid.

20. The artificial intelligence-based method of claim 16, wherein generating the inquiry response comprises:
matching the at least a nutritional need of the user with at least a nutritional need pertaining to at least a prior user;
pairing the at least a prior user with the user as a function of the match; and
identifying the at least a prior user in the inquiry response to the user.
